(19) United States
(12) Patent Application Publication
(10) Pub. No.: US 2026/0240688 A1
(43) Pub. Date: Aug. 20, 2026

(54) BANDAGES OR WOUND DRESSINGS AND METHODS OF MAKING SAME

(71) Applicant: Pellis Innovations Limited, Edinburgh (GB)

(72) Inventor: Brian James McCormack, By Kirkcaldy (GB)

(73) Assignee: Pellis Innovations Limited, Edinburgh (GB)

(21) Appl. No.: 19/470,651

(22) PCT Filed: Mar. 8, 2024
(86) PCT No.: PCT/GB2024/050615

§ 371 Date: Sep. 29, 2025

(30) Foreign Application Priority Data
Mar. 30, 2023 (GB) 2304756.6

(51) Int. Cl. A61F 13/01; A61F 13/00; D04B 1/16; D04B 1/22; D04B 21/20

(52) U.S. Cl. CPC A61F 13/01008; A61F 13/01025; D04B 1/16; D04B 1/22; D04B 21/20; A61F 2013/00093; A61F 2013/00238; D10B 2201/02; D10B 2331/04; D10B 2331/10; D10B 2509/022

(57) ABSTRACT

The present disclosure relates to a bandage or a dressing with a quick release mechanism wherein said bandage or dressing is made from a woven material comprising at least one pull or draw-thread that runs through the material across the bandage or dressing, from any one side to another and to a method for removing at least a part of a bandage or dressing by pulling on at least one pull or draw-thread provided in a knitted material.

The present invention relates to a bandage or a dressing with a quick release mechanism wherein said bandage or dressing is made from a woven material comprising at least one pull or draw-thread that runs through the material across the bandage or dressing, from any one side to another or within the material to create an opening.

### INTRODUCTION

A dressing is a sterile pad or compress applied to a wound to promote healing and protect the wound from further harm, it is designed to be in direct contact with the wound, as distinguished from a bandage, which is most often used to hold a dressing in place or, alternatively, to provide support to/restrict the movement of a part of the body.

Typically, a dressing is used to help to seal a wound and so expedite the clotting process, thus stopping bleeding; provide a barrier to minimise the risk of infection and mechanical damage; absorb exudate so containing the wound and preventing maceration; medicate the wound by containing medication in close contact with same; and obscure a healing wound from the patient, so reducing psychological stress.

Ultimately, the aim of a dressing is to promote optimised wound healing by providing a sterile environment that facilitates efficient epithelialization, helping the wound to heal more quickly, and reduces scarring.

Hitherto, bandages or dressings, commonly, have been made of cotton or crepe. Modern dressings are made from dry or impregnated gauze, plastic films, gels, foams, hydrocolloids, alginates, hydrogels, polysaccharide pastes, granules and beads. They all provide different physical environments suited to different wounds. Often dressings are impregnated with analgesics to reduce pain.

As mentioned, bandages are often used to keep a dressing in place or simply attach a dressing to an awkward space, but they can also be preferred when the application of compression would facilitate healing such as after a burn injury where the control or reduction of swelling is favoured.

Additionally, bandages are used to support the healing of sprains and strains, as they provide good compression to injured areas. The woven material of a bandage allows the skin to breathe, and they have the added environmental advantage of being washable and reusable.

Elasticated bandages are known and favoured because their tension, upon stretch, allows a user to position the bandage close to a patient's body contours. These bandages are often used for securing dressings in place, especially on limbs or where compression is favoured. Such bandages are lightweight, fray-resistant and breathable. These are usually made with synthetic materials and are provided in the form of an endless roll of material. The comfort of elasticated bandages makes them especially suitable for retaining dressings on difficult parts of the body or for soft tissue injury, but their use as the only means of applying pressure to an oedematous limb or to a varicose ulcer is limited, since the pressure they exert is inadequate. Thus, where significant levels of compression are required, non-elastic bandages are used such as for venous leg and diabetic foot ulcers.

Tubular bandages are also available and differ in length/diameter according to the function required of them. For example, some are used under orthopaedic casts, and some are used for protecting areas to which creams or ointments (other than those containing potent corticosteroids) have been applied.

A problem with the above-mentioned dressings and bandages is the ease with which they can be removed, particularly from sensitive skin or difficult parts of the body. Clearly, during the course of healing, whether of a wound or a sprain/fracture, a number of dressings or bandages may have to be applied which means a number of dressings or bandages have to be removed. Often dressings or bandages are removed by peeling or unwrapping, respectively, which can cause pain and trauma and mechanical damage to the skin.

It is an aim of the present invention to reduce any trauma associated with the removal of a bandage or dressing, including a conventional tubular bandage, whilst maintaining the required integrity of the wound.

### STATEMENTS OF INVENTION

Accordingly, a first aspect of the present invention comprises a bandage or dressing made from a woven material wherein at least one thread of said woven material provides at least one pull or draw-thread that runs through the material across the bandage or dressing, from any one side to another, or within the material to create an opening whereby, when said thread is pulled, the bandage or dressing is torn apart along the length of said pull or draw-thread thereby separating the bandage or dressing into at least two parts or creating an opening in same.

In a further preferred embodiment of the invention said at least one pull or draw-thread is not an integral part of the bandage or dressing weave but, instead, represents a separate knit which is then attached or sewn onto a piece of bandage fabric. Specifically, and most advantageously or preferably, the pull or draw-thread is first knitted as a piece of fabric comprising a pull or draw-thread and then this piece is, ideally, turned through a selected angle, such as 90 degrees, and then attached or sewn onto another piece of fabric intended to form a bandage. Thus, the pull or draw-thread does not represent a stitch, such as for example a pillar stitch, within the piece of fabric intended to form a bandage. This is advantageous as it means the pull or draw-thread can be sewn into the bandage material to cross same in any desired direction, thus allowing the bandage material and the way it can be dismantled, or torn into pieces, to be entirely customised to a user's requirements. This versatility permits the production of a plurality of different bandages each one having a selected number of pull or draw-threads traversing said bandage in a particular manner to form a series, or at least one series, that is perfectly suited to the function it is to perform.

In this way a bandage or dressing may be constructed, in its most simple form, with a plurality of pull or draw-threads that each run across the bandage in the same, but selectively spaced, direction.

In an alternative form a bandage or dressing may be constructed, in a more complex form, with a plurality of pull or draw-threads that each run across the bandage in different, but selectively spaced, directions. For example, the latter embodiment of the invention may be illustrated in the form of a tubular bandage that has one, or a plurality of, pull or draw-thread(s) running across the bandage (vertical and/or horizontal) in a regular or irregular, zig-zagging or meandering, fashion which fashion traces the shape of the limb to which the bandage or dressing is attached thus, upon pulling on the pull or draw-thread, the bandage is dismantled in a manner that represents the shape of the limb and so causes the least discomfort to the patient.

In yet a further embodiment of the invention said pull or draw-thread runs through the material in the form of a circle, ellipse, square, rectangle, triangle, indeed any preferred but continuous shape so that, upon pulling the thread it creates an opening corresponding to the shape of the pulled thread, such as an inspection opening, in the dressing or bandage.

In a further preferred embodiment of the invention the pull or draw-thread is produced by a weft knitting process and, ideally, the piece of fabric intended to form the bandage is also produced by a weft knitting process. Thereafter, a further knitting process takes place to add the pull or draw-thread knit to the piece of fabric intended to form the bandage. In this latter process an endless length of bandage may be produce or an additional knit incorporated to create a tube of bandage. Essentially, in our invention, the pull or draw-thread is a joining thread, and it is physically pulled out of the fabric to separate the joined pieces of bandage. Most typically, the pull or draw-thread is an integral part of the final bandage and runs, ideally (horizontally) across the weft knit fabric.

In a preferred embodiment of the invention said bandage or dressing is provided with at least one pull or draw-thread that runs through the material and across the bandage, from any one side to another, in either a horizontal, vertical or diagonal direction. Most ideally the said thread runs in a direction at either 90° or 45° to the longitudinal axis of the bandage, most suitably at 90°, whereby a user can separate parts of the bandage. Ideally, a plurality of said pull or draw-threads are provided whereby one can, sequentially, pull upon such threads to remove, piece-by-piece, sections of bandage to ultimately remove the entire bandage. In this embodiment of the invention the distance between such threads, along the length of the bandage or dressing will, generally equate to the diameter of an average leg or arm, although bandages or dressings suited for different sized limbs may be provided, small/medium/large, and characterised by the spacing between said threads.

In a preferred embodiment of the invention said bandage or dressing is provided with at least one pull or draw-thread that runs through the material and across the dressing, from one side to another, in either a horizontal, vertical or diagonal direction. In certain embodiments a plurality of pulls or draw-threads are provided that run through the material across the dressing, from one side to another, in a number of directions including a horizontal, vertical or diagonal direction. In this way, a user can determine how to remove the dressing by pulling upon a selected one or more of the threads to separate the dressing, most advantageously, having regard to the nature of the underlying wound.

In an alternative embodiment of the invention, said bandage or dressing is provided with at least one pull or draw-thread that runs through or within the material to create a desired but continuous shape representing a window, such that when the thread is pulled a window, corresponding to the path of travel of said pull thread through or within said material, is created.

In yet a further preferred embodiment of the invention, two adjacent pull or draw-threads are provided, this arrangement eases the ability of a user to grip hold of the threads and pull the bandage or dressing apart. Preferably three or more adjacent threads are chosen but whilst this facilitates use, it does increase the material costs and wastage.

Preferably, the pull or draw thread is of a different colour to the material of the rest of bandage or dressing for identification purposes.

More preferably still, the pull or draw threads comprise a HT (High Tenacity) yarn, ideally a polyester yarn. Yet more preferably, said HT yard has a linear density of at least 235 dtex (deci-tex), i.e., a mass of at least 235 g per 10,000 meters of yarn. Most preferably the HT yarn has a linear density of about 235 dtex.

In yet a further preferred embodiment, the bandage or dressing is conventionally woven using weft and warp threads and, ideally, the pull or draw-thread is either a weft or a warp thread. Ideally, the pull or draw-thread is introduced during manufacture to the weft (for flat bandages) or the warp (for tubular bandages/dressings) of the material preferably a plurality of the pull or draw-thread are created in each bandage or dressing, thus constituting a number of tear or break points for separating one part of the bandage or dressing from another. Most preferably, the bandage or dressing is conventionally woven using weft threads and the pull or draw-thread is woven using a weft thread. However, when the bandage or dressing is joined to the knit comprising the pull or draw-thread one of the two materials is turned through a selected angle, such as 90°, before stitching same together.

According to a second aspect of the invention there is provided a method for making a bandage or dressing comprising:
i) knitting within a section of fabric at least one pull or draw thread;
ii) knitting or obtaining a section of bandage or dressing;
iii) sewing or attaching the knitted fabric of part i) to the section of part ii); thereby making a bandage or dressing including within same at least one pull or draw thread that runs through the material across the bandage or dressing, from any one side to another or within the material to create an opening in same.

In a preferred method of the invention, said knitted section of part i) is woven using weft threads.

In yet a further preferred method of the invention, said knitted section of part ii) is woven using weft threads.

More preferably still, prior to undertaking the sewing or attaching of part iii) the knitted section of part i) or ii) is rotated through a selected angle, such as 90°, with respect to the non-rotated section of part ii) or i), respectively.

According to a further aspect of the invention there is provided a method for removing at least a part of a bandage or dressing comprising pulling on at least one pull or draw-thread provided in a woven material constituting said bandage or dressing wherein said thread runs through the material and across the bandage or dressing, from any one side to another, or within the material to create an opening whereby, when said thread is pulled, the bandage or dressing is torn apart along the length of said pull or draw-thread thereby separating the bandage or dressing into at least two parts or creating an opening in same.

In use, when the pull or draw-thread is activated, by physically pulling on same, the thread is removed from the woven structure and the bandage or dressing separates along the thread line, the remainder of the bandage can then be eased away from the wound or, alternatively, a further pull or draw thread is pulled, thus removing another part of the bandage and so facilitating the removal of any remaining bandage or dressing.

In another use, when the pull or draw-thread is activated, by physically pulling on same, the thread is removed from the woven structure and an opening is created in the bandage or dressing, thus allowing access to the underlying wound or further dressing, optionally, the remainder of the dressing can then be eased away from the wound by pulling on a further pull or draw thread, thus removing another part of the dressing and so facilitating the removal of any remaining bandage or dressing.

This gentle and sequential removal of parts of the bandage or dressing reduces pain, trauma (of patient and nurse) and any mechanical skin damage. With lesser skin damage the present invention also reduces the potential for infection which could lead to further complications. The average time to change a wound dressing is 18 minutes. With the avoidance of time-consuming unwrapping, this system will significantly reduce the time taken to change a dressing. Additionally, the invention enables this efficient removal by someone who can't necessarily reach their feet and or toes to remove a bandage or dressing, such as, in one example, a roll-down tubular bandage. The invention enables them to pull the string and decrease removal time to seconds as the bandage falls away. Equally, the utility of removal of a bandage or dressing from an infirm or immobile patient is facilitated when one can break the bandage or dressing across sections, instead of unwinding a bandage or tearing across the adhesive of a dressing.

Preferred features of each aspect of the invention may be as described in connection with any of the other aspects.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Examples of bandages and dressings made according to the present invention will now be described in relation to the accompany drawings, in which:

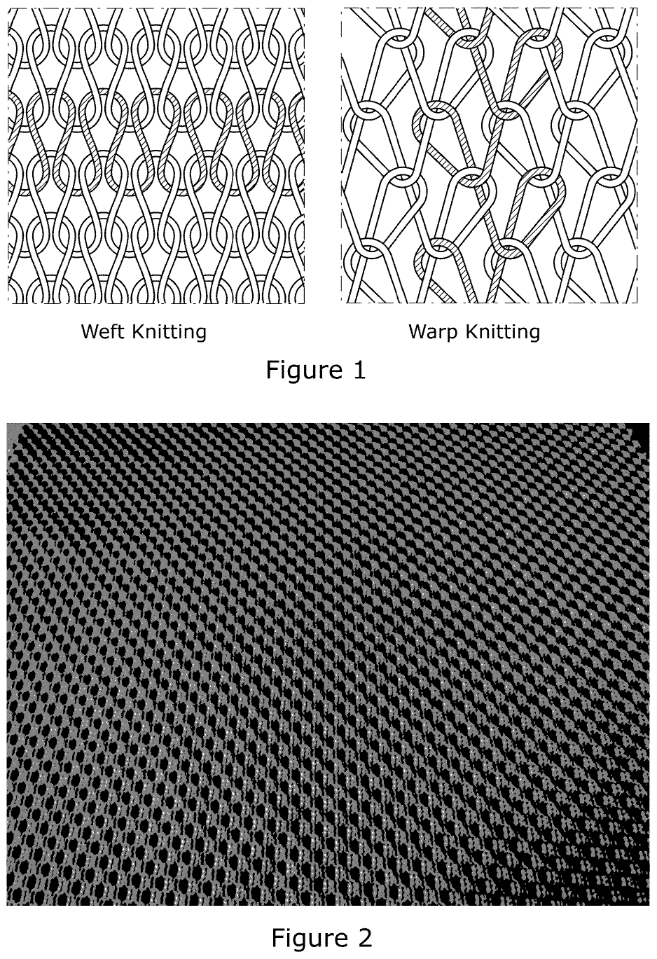

FIG. 1 shows a plan view of two types of weaving, namely weft and warp, used in the present invention;

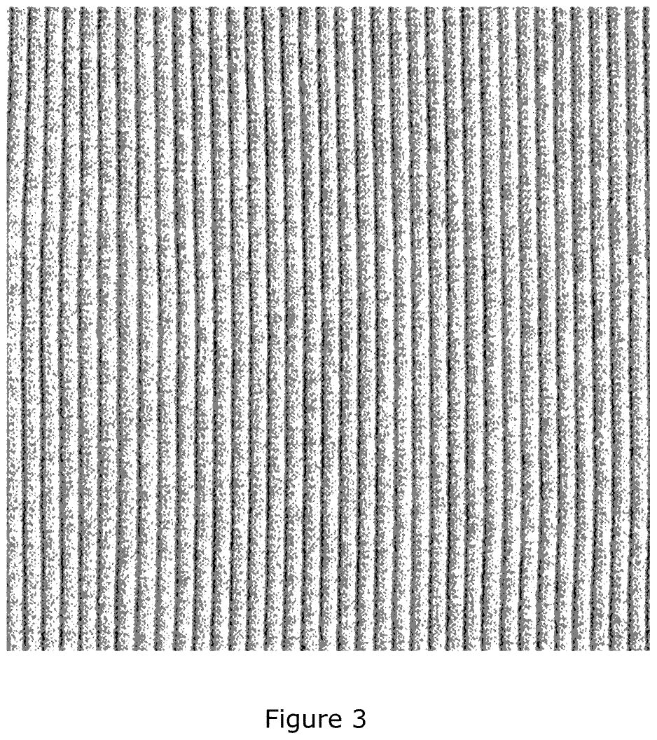

FIG. 2 shows a perspective view of a small hole warp knitted mesh fabric;

FIG. 3 shows a perspective view of a 1×1 weft knitted rib fabric;

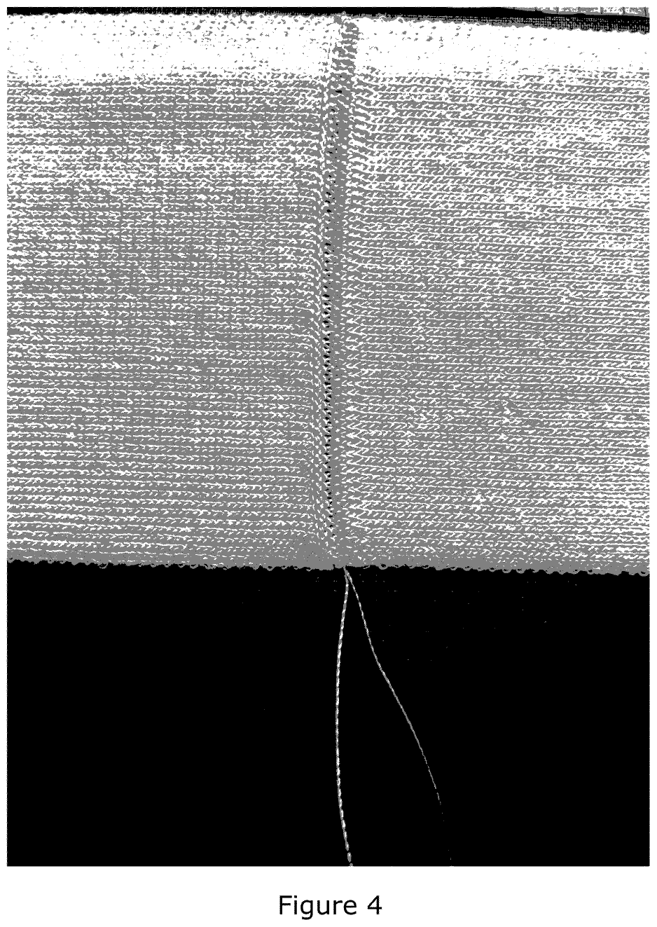

FIG. 4 shows a perspective view of a bandage join according to the invention with two release or removal threads;

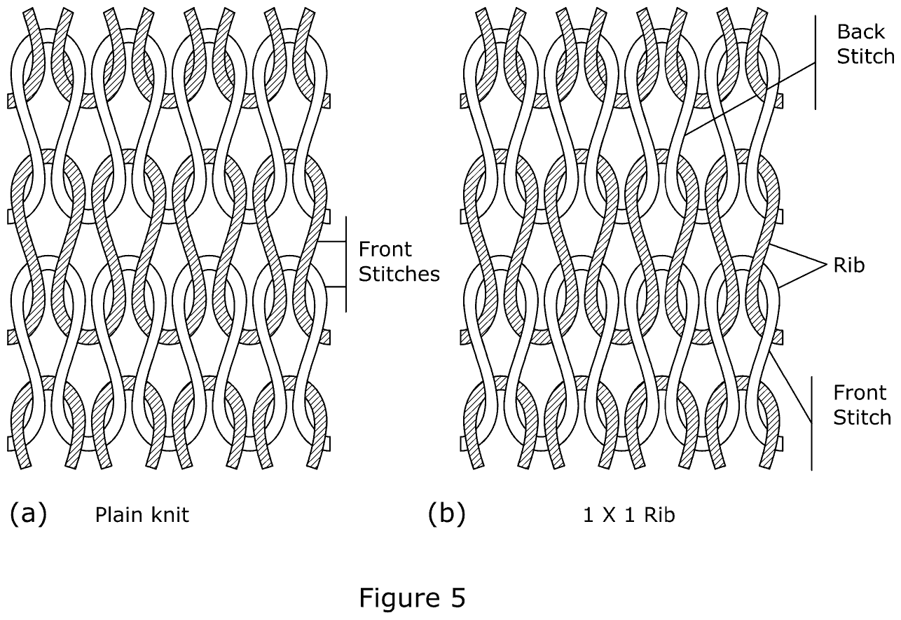

FIG. 5 shows a plan view of plain knitting and 1×1 rib knitting, used in the present invention;

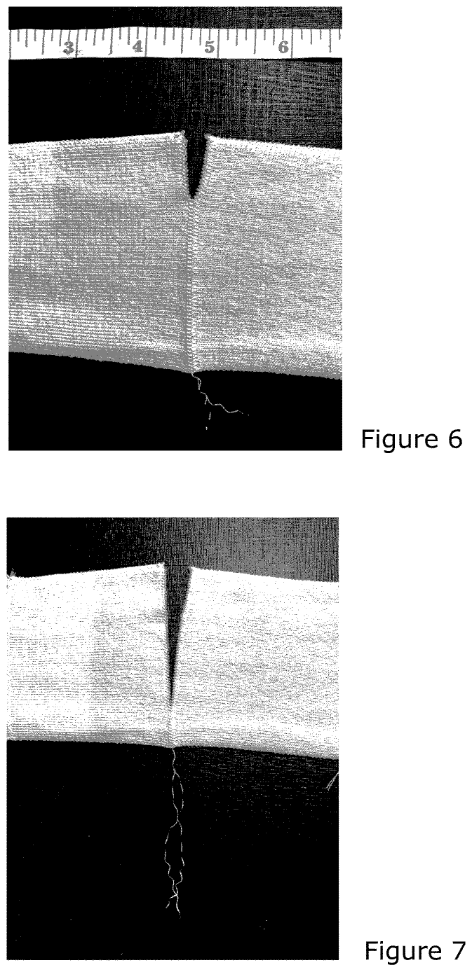

FIG. 6 shows a perspective view of a bandage join according to the invention with two threads partial pulled for release;

FIG. 7 shows a perspective view of a bandage join according to the invention with two threads further pulled for release;

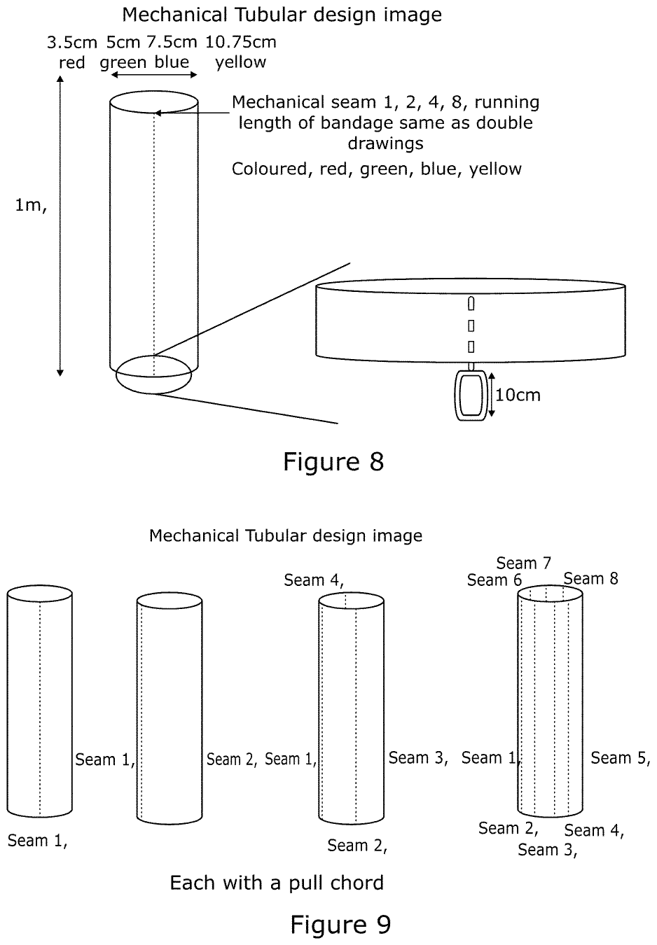

FIG. 8 shows a schematic view of a tubular bandage according to the invention including a pull or draw-thread;

FIG. 9 shows a schematic view of a tubular bandage according to the invention including a plurality of longitudinal pull or draw threads.

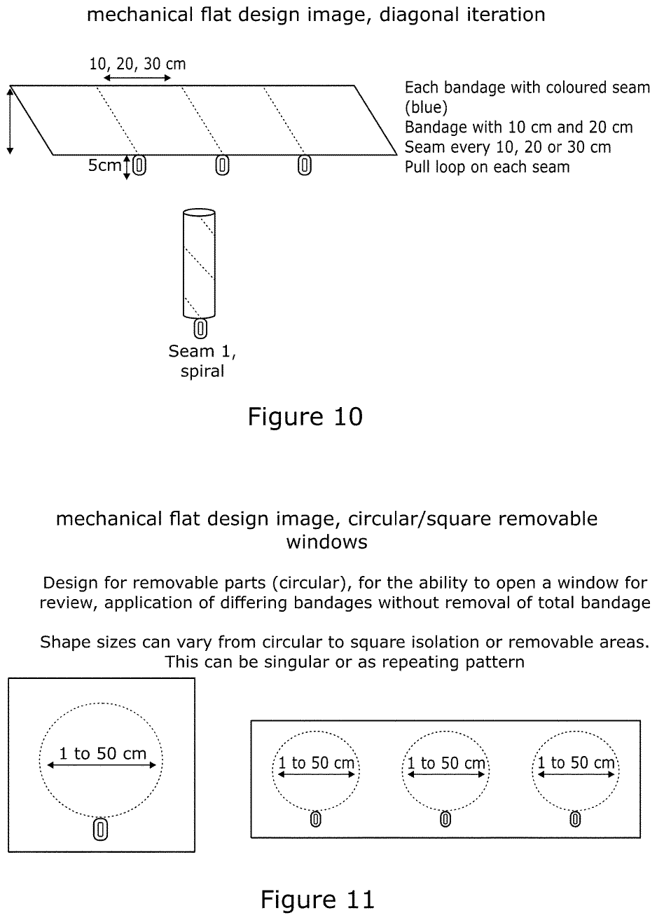

FIG. 10 shows a schematic view of an endless bandage according to the invention including a plurality of pull or draw-thread running diagonally across same; and

FIG. 11 shows a schematic view of a dressing (A) and a part of an endless bandage according to the invention including at least one circular pull or draw thread within same, each defining the outline of a circular opening.

Referring firstly to FIG. 1 there is shown a diagrammatical illustration of a plain weft knit structure and a tricot warp knit structure of the type typically used in the making of bandages.

### FLAT TYPE BANDAGES/DRESSINGS

The conventional (flat) bandage prototype is produced by weft knitting using a V-bed flat machine. 1×1 rib is produced by two sets of needles being alternately set or gated between each other. Relaxed 1×1 rib is theoretically twice as thick and half the width of a plain fabric but it has twice as much width wise recoverable stretch. The 1×1 rib is balanced by alternate weaves of face loops in each side, it therefore lies flat without curl when cut. It is a more expensive fabric to produce than a plain-woven fabric and is a heavier knit structure, the rib machine also requires a finer yarn than a similar gauge plain machine. The product of this knitting process is shown in FIG. 3.

A bandage or dressing according to the present invention is produced by providing or including a pull or draw-thread which is knitted into the weft (horizontal) at predetermined spaced intervals such as every few inches. Prototypes have been knitted, for example, using ecru coloured Ne 1/38 cotton doubled with elastane yarn in a 1×1 rib construction using a 16 gauge machine, 3″ wide with the pull/draw-thread introduced every 3″ using 2 ends of HT (#High Tenacity) polyester yarn.

### TUBULAR BANDAGES/DRESSINGS

A tubular dressing is usually produced by warp knitting using a DNB (Double Needle Bar) Raschel machine. According to the present invention the pull/draw-thread is knitted into the warp knit mesh structure longitudinally (along the wale). Prototypes have been knitted on a DNB Raschel warp knitting machine with 20 cpi (courses per inch) and 42 cpi using 167 dtex textured polyester yarn.

FIG. 4 shows a further embodiment of the present invention where the break apart uses a two pull or draw-threads. This is realised using the weft knitted method of manufacture. The conventional (flat) bandage is produced by weft knitting using a 12-gauge flatbed Shima Seiki jacquard rib transfer machine. The weft knitted structure is a 1×1 rib fabric, 3 inches wide, whereby the pull or draw-thread is knitted into the weft (horizontal) every, say 3 inches or so. The break points are knitted using a single course (row) of HT (High Tenacity) polyester yarn with a count (thickness) of 235 dtex. The prototype 12-gauge weft knitted 1×1 rib fabric is knitted using a cotton yarn with a count (thickness) of Ne 1/24 together with an elastane yarn with a count (thickness) of Ne 1/44. The elastane yarn increases the amount of stretch and recovery in the fabric.

In the bandage illustrated in FIG. 4 the pull or draw-thread is knitted every 3″, or so, into the 1×1 rib structure using two ends of HT polyester 235 dtex. The weft knit rib transfer machine allows the pull or draw-thread to be transferred and then knitted on half of the allocated needles making it easier to withdraw the thread from the knitted structure and reducing the length of draw-thread being removed. The pull or draw-thread comprises two threads to improve the ability of a user to pull on the threads.

The bandage in FIG. 4 is knitted on a 16-gauge v bed weft knit flat machine, the draw thread was knitted as a single course (row) on both knitting beds, front and back which creates a zig zag pattern and an un-natural path for the removal of the thread. It also creates a lengthy amount of draw-thread to pull out or remove.

Alternatively, a bandage can be knitted using a 12-gauge weft knit rib transfer machine the cotton and elastane yarns are knitted on both beds. The front bed stitches are then transferred to the back bed followed by a slack course (row). There is then a single row of pull or draw-thread on the back bed which uses only half the needles of the 16 gauge version and all the needles are presented level so the thread removes easily. Because there is no draw-thread moving from front to back needles the amount of yarn is substantially reduced by more than half. The front bed needles are then picked up with the first cotton/elastane set up course (row). As it is also 12 gauge the knitted loops are larger and the needle field smaller all helping to make it easier for the draw-thread to be removed.

FIG. 5 shows the diagrammatic knitting involved in the 1×1 ribbed bandage. In this Figure the rib is knitted in between a front and a back stick and matches the pattern in the plain knit.

FIGS. 6 and 7 show the pull or draw-thread being removed and the bandage or dressing material splitting apart as the thread un-laces adjacent parts of the bandage or dressing.

Ideally, although only shown in FIGS. 1 and 5, the pull or draw-thread in both the weft and warp knitted structures will be a different colour to the main bandage fabric for ease of identification and so use.

FIG. 8 is an illustration of the invention when used to make a tubular bandage or dressing. In this instance a single pull or draw-thread is sewn into or attached to the fabric used to make the tubular bandage along a longitudinal axis of same, thus providing for a tear that will run the entire longitudinal length of the tubular bandage and so facilitate the removal of same.

Similarly, in FIG. 9, there is shown a tubular bandage or dressing comprising a plurality of pull or draw-threads sewn into or attached to the fabric used to make the tubular bandage, each one existing along a different longitudinal axis of the fabric, thus providing for a plurality of tears that will run the entire longitudinal length of the tubular bandage and so facilitate the sequential removal of sections of the bandage.

Although not illustrated. The tubular bandage may comprise a plurality of pull or draw-threads, suitable spaced from each other but crossing the bandage in any number of directions whereby, in use, a specific segment can be removed prior to removing another segment thus accommodating removal of the bandage from an appendage or a particular shape such as a bent elbow.

In FIG. 10 there is illustrated an endless bandage comprising a plurality of pull or draw threads (dark seams), roughly 10, 20 or 30 cm apart, according to a user's requirements, and having attached to one or more of said pull or draw threads, at an end of said bandage, a loop whereby a user can hold the loop to pull upon the pull or draw thread in order to separate said bandage along said selected pull or draw thread. Also in FIG. 10 a tubular bandage is shown including a plurality of pull or draw threads (dark seams), roughly 10, 20 or 30 cm apart, according to a user's requirements, and having attached to one or more of said pull or draw threads, at an end of said bandage, a loop whereby a user can hold the loop to pull upon the pull or draw thread in order to separate said bandage along said selected pull or draw thread.

In FIG. 11 there is illustrated a dressing, left hand side, having a pull or draw thread within the material of same, wherein said pull or draw thread traces a selected shape, in this instance a circle, through or within said dressing so that when a user pulls upon same a window, corresponding to the shape provided by the pull or draw thread, is created. Also in FIG. 11, right hand side, there is shown either a larger dressing or a part of an endless bandage where a number of pull or draw threads are provided in a spaced manner to create a series of adjacent shapes, in this instance circles, so a user can select where along the length of the dressing to provide an opening by pulling on one or more of said pull threads.

1. A bandage or dressing made from a woven material wherein at least one thread of said woven material provides at least one pull or draw-thread that runs through the material and across the bandage or dressing, from any one side to another, or within the material to create an opening whereby, when said thread is pulled, the bandage or dressing is torn apart along the length of said pull or draw-thread thereby separating the bandage or dressing into at least two parts or creating an opening in same.

2. The bandage or dressing according to claim 1 wherein said at least one pull or draw-thread is provided in a separate knit which is then attached or sewn onto a piece of bandage or dressing fabric.

3. The bandage or dressing according to claim 1, comprising a plurality of pull or draw-threads traversing said bandage in a particular manner to form at least one series, suited to the function the bandage or dressing is to perform.

4. The bandage or dressing according to claim 3 wherein a plurality of said series are repeated along the length of said bandage or dressing.

5. The bandage or dressing according to claim 1, wherein a plurality of pull or draw-threads are provided each one running across the bandage in the same, but selectively spaced, direction.

6. The bandage or dressing according to claim 1, wherein a plurality of pull or draw-threads are provided each one running across the bandage in a different, but selectively spaced, direction with respect to any one or more other pull or draw-thread.

7. The bandage or dressing according to claim 1, wherein the bandage or dressing is a tubular bandage or an elongate length of bandage that has one, or a plurality of, pull or draw-thread(s) running across the bandage (vertical and/or horizontal) in a regular or irregular zig-zagging or meandering fashion which traces the shape of the limb to which the bandage is to be attached.

8. The bandage or dressing according to claim 1, wherein the bandage or dressing has at least one pull or draw thread within same defining an opening in the bandage or dressing.

9-13. (canceled)

14. The bandage or dressing according to claim 1, wherein said bandage or dressing is provided with at least one pull or draw-thread that runs through the material and across the bandage or dressing, from one side to another, in either a horizontal, vertical, diagonal, zig-zag or meandering direction.

15. The bandage or dressing according to claim 1, wherein said bandage or dressing is provided with at least one pull or draw-thread that runs through or within the material tracing a shape that is to provide an opening in said bandage or dressing.

16. The bandage or dressing according to claim 1, wherein a pair of adjacent pull or draw-threads are provided that run through or within the material either i) across the dressing, from any one side to another, in a number of directions including a horizontal, vertical or diagonal direction, or ii) tracing a shape that constitutes an opening within the dressing or bandage.

17. The bandage or dressing according to claim 1, wherein a plurality of said pulls or draw threads are provided next to each other.

18. The bandage or dressing according to claim 1, wherein said thread(s) run(s) at least partially in a direction at either 90° or 45° to the longitudinal axis of the bandage or dressing.

19. (canceled)

20. The bandage or dressing according to claim 1, wherein the spacing between said plurality of pulls or draw threads is calibrated having regard to the size of a body part to be bandaged.

21-22. (canceled)

23. The bandage or dressing according to claim 1, wherein the pull or draw thread comprise a HT (High Tenacity) yarn.

24. The bandage or dressing according to claim 23 wherein, ideally said yarn has a linear density of at least 235 dtex.

25-26. (canceled)

27. A method for making a bandage or dressing comprising:
i) knitting within a section of fabric at least one pull or draw thread;
ii) knitting or obtaining a section of bandage or dressing;
iii) sewing or attaching the knitted fabric of part i) to the section of part ii); thereby making a bandage or dressing including within same at least one pull or draw thread that runs through the material across the bandage or dressing, from any one side to another or within the material to create an opening.

28. The method according to claim 27 wherein said knitted section of part i) is woven using weft threads and/or said knitted section of part ii) is woven using weft threads.

29. The method of claim 27, wherein prior to undertaking the sewing or attaching of part iii) the knitted section of part i) or ii) is rotated through a selected angle such as 90° with respect to the non-rotated section of part ii) or i), respectively.

30. A method for removing at least a part of a bandage or dressing comprising pulling on at least one pull or draw-thread provided in a woven material constituting said bandage or dressing wherein said thread runs through or within the material and either i) across the bandage or dressing, from any one side to another, or ii) within the material to create an opening;
whereby, when said thread is pulled, the bandage or dressing is torn apart along the length of said pull or draw-thread thereby separating the bandage or dressing into at least two parts or creating an opening in same.
